(19) United States
(12) Patent Application Publication
(10) Pub. No.: US 2009/0012375 A1
(43) Pub. Date: Jan. 8, 2009

(54) Implantable sensor and connector assembly

(76) Inventor: Kyriacos Pitsillides, Sacramento, CA (US)

Correspondence Address: Stephen B. Salai, Esq.;Harter, Secrest & Emery LLP, 1600 Bausch & Lomb Place, Rochester, NY, 14604-2711, US

(21) Appl. No.: 12/215,912

(22) Filed: Jun. 30, 2008

Related U.S. Application Data
(60) Provisional application No. 60/958,077, filed on Jul. 2, 2007.

(51) Int. Cl. A61B 5/07; A61B 5/03

(52) U.S. Cl. 600/302; 600/561

(57) ABSTRACT

An apparatus is disclosed that provides an implantable sensor assembly configured with signal conditioning circuitry positioned between a sensor and connector to form a unitary liquid impervious flexible structure. The connector is adapted to be detachably connected to an implant service unit, which service unit provides battery power, control functions and wireless communication with a base station. The sensor unit is modular in nature and allows for easy exchange with the implant service unit and other sensors.

### CROSS-REFERENCE TO RELATED APPLICATIONS

The present application claims priority under 35 USC § 119 (e) from U.S. provisional application Ser. No. 60/958,077 field Jul. 2, 2007 and titled “Implantable Sensor and Connecter Assembly”.

### STATEMENT REGARDING FEDERALLY SPONSORED RESEARCH OR DEVELOPMENT

Not applicable.

### REFERENCE TO A “SEQUENCE LISTING”

Not applicable.

### BACKGROUND OF THE INVENTION

1. Field of the Invention

The present invention relates to implantable telemetric systems, and more particularly it relates to a system and method for implanting and detachably connecting a sensor to a telemetric implant in an enhanced fashion that avoids high impendence and other problems inherent in such systems in the past.

2. Background of the Invention

In conducting scientific studies and providing treatment implantable sensors are used for both animal research and human monitoring, and these sensors exist in a variety of forms. Normally these sensors, such as piezoresistive blood pressure or temperature sensors, or bioelectric signal sensors are either connected directly to the implant, or are connected via an implantable connector. Ordinarily, the implant contains circuitry that provides power and signal conditioning to the sensors.

Implantable systems with easily detachable connectors add a high degree of flexibility to the system by allowing the quick replacement of a damaged sensor or the selection of a sensor with different characteristics. There are though a number of problems that are introduced by the use of an implantable connector to connect physiological sensors to an implant. The majority of these problems are caused by the high impedance inherent in these sensors. This high impedance when affected by the implantation connector system causes DC drift, alteration of the true value of the sensor measurement, etc.

FIG. 1 is a schematic diagram depicting a prior art configuration of senor connector assembly 20 with a sensor 21 and connector assembly 23 and 24 that connects sensor 21 to a patient monitor 29 as indicated by U.S. Pat. No. 5,568,815. The amplifier and temperature compensation circuitry 27 are located before the patient monitor display 29. This system is not suitable for implantation, since the electrical connector assembly 23 and 24 does not have any provisions for providing a low impedance interface to the sensor connector assembly. Sensor 21 lead 32 comes out of the body 30 through skin 31 before the first connection is made and therefore the electrical connector does not come into contact with body fluids. This will not be the case in an implantable system and this sensor assembly would not be usable in such a case.

Similarly, patent application US2007/0106165A in FIG. 2, discloses a similar sensor assembly 41 that incorporates the amplifier and temperature compensation circuitry 45 in connector 47 of connector set 46 and 47 outside the patient's body 49 before it connects to monitor display 51. Again this arrangement will only work in a non-implantable situation, were the connector is outside the body and not in contact with body fluids.

Interfacing a physiological sensor that has high impedance such as a piezoresistive blood pressure sensor to an implant via a connector, which is then placed into an animal may cause small changes in the value of the piezoresistive elements comprising the sensor. These small impedance changes are due to moisture trapped inside the connector which when placed inside the warm animal body cause the microenvironment inside the connector to change. These changes to the sensor impedance are small, but the normal changes to the impedance of the sensor to changes in blood pressure are also small, in the order of 2-3% for a full scale change of physiological range blood pressure. These small changes in impedance can create significant errors in readings taken by the sensor. These errors are of such an extent that readings obtained by the sensor will have no useful value.

Such changes due to the moisture effect manifest as a slow and unpredictable drift of the pressure signal with either a positive and/or a negative sign. Although there are methods that can be used to prevent such moisture generation and buildup, such as filling the connector with silicone oil, or mineral oil, these measures are time consuming and not always able to remain viable. Also they might impede with the normal use of the connector insulation.

Thus what is needed is a fully implantable modular biotelemetric monitoring system that does not have the disadvantages of the prior art that made it impossible to provide detachable piezoresistive that did not suffer form signal degradation.

### SUMMARY

Thus, it is an objective of the present invention to provide a fully implantable modular biotelemetric monitoring system with detachable connectors to all allow the changing of sensors.

These problems are alleviated by the use of an impedance converter circuit that converts the high sensor impedance to very low impedance prior to the signal passing through a connector junction.

The present invention achieves these and other objectives by providing: an implantable telemetric sensor system having: a) a sensor assembly including a sensor, signal conditioning circuitry and a connector all in a sealed liquid impervious package; b) an implant service package with a power supply to power the implant service unit, a microcontroller to control operation of the implant service unit, memory for storing software to run the implant service unit with the microcontroller, a transceiver for communicating with a base unit and a connector capable of forming a liquid impervious detachable electrical connection with the sensor connector of the sensor assembly; and c) wherein when the sensor assembly is implanted inside a living biological system and the sensor is connected to monitor a function of the biological system and the connector is attached to a connector of the implant service package.

In a further aspect of the present invention the conditioning circuitry includes circuitry for amplifying signals generated by the sensor. In yet another aspect of the present invention the conditioning circuitry includes temperature compensating circuitry. In yet another aspect of the present invention the conditioning circuitry includes circuitry for changing the impedance of signals generated by the sensor. Yet still another aspect of the present invention the sensor assembly is pressure sensor that uses a piezoresistive surface for sensing pressure.

In a further aspect of the present invention it provides an implantable sensor assembly having: a) a sensor in electronic communication with conditioning circuitry and the conditioning circuitry is electrical communication with a connector, and wherein the sensor, the conditioning circuitry and the connector are joint as one unit in a liquid impervious sealed package; and b) the connector of the sensor assembly is configured to form a detachable liquid impervious detachable connection with a connector of an implant service unit.

### BRIEF DESCRIPTION OF THE DRAWINGS

The invention will be better understood by an examination of the following description, together with the accompanying drawings, in which:

FIG. 1 a schematic diagram of a prior art sensor connector assembly array;

FIG. 2 a schematic diagram of another prior art sensor connector assembly array;

FIG. 3 a diagram of a preferred embodiment of the sensor connector assembly array of the present invention;

FIG. 4 is a plan view of a preferred embodiment of a sensor assembly made according to the present invention;

FIG. 5 is a plan view of another sensor assembly made according to the present invention; and

FIG. 6 is a plan view of the complete components of the system of the present invention.

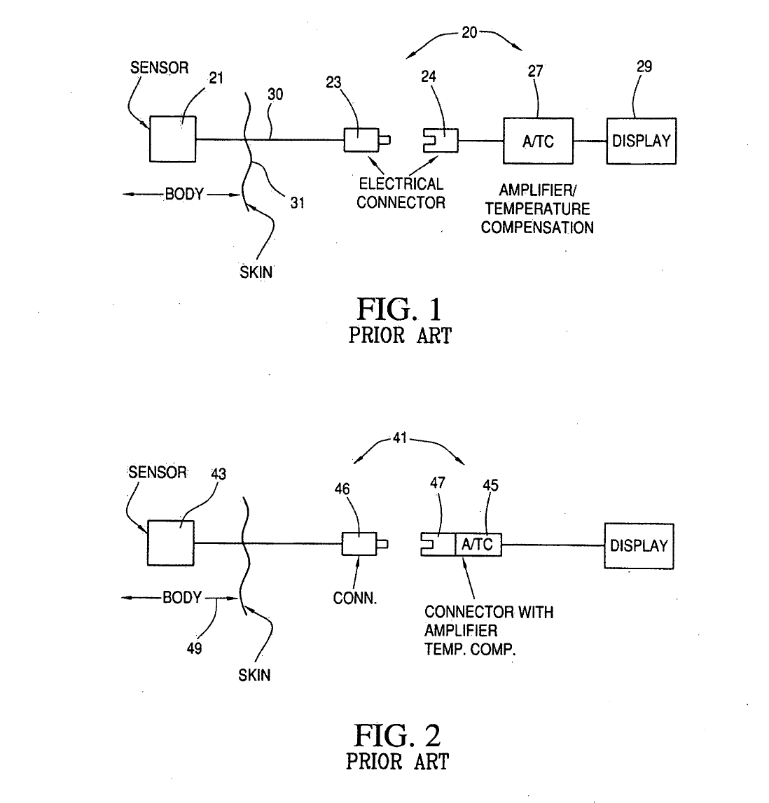

### DETAILED DESCRIPTION OF THE PREFERRED EMBODIMENT

Interfacing a physiological sensor that has high impedance such as a piezoresistive blood pressure sensor to an implant via a connector, which is then placed into an animal may cause small changes in the value of the piezoresistive elements comprising the sensor. These small impedance changes are due to moisture trapped inside the connector which when placed inside the warm animal body cause the microenvironment inside the connector to change. These changes to the sensor impedance are small, but the normal changes to the impedance of the sensor to changes in blood pressure are also small, in the order of 2-3% for a full scale change of physiological range blood pressure.

Such changes due to the moisture effect manifest as a slow drift of the pressure signal with either a positive and/or a negative sign. Although there are methods that can be used to prevent such moisture generation and buildup, such as filling the connector with silicone oil, or mineral oil, these measures are time consuming and not always able to remain viable. Also they might impede with the normal use of the connector insulation.

The impedance converter circuit built into the sensor of the present invention between the sensor and its connector provides amplification and conversion of the differential signal into a unipolar signal and also converts the high impedance (about 3 kOhm to 100 kOhm) to low impedance (about 0.1 Ohm or less).

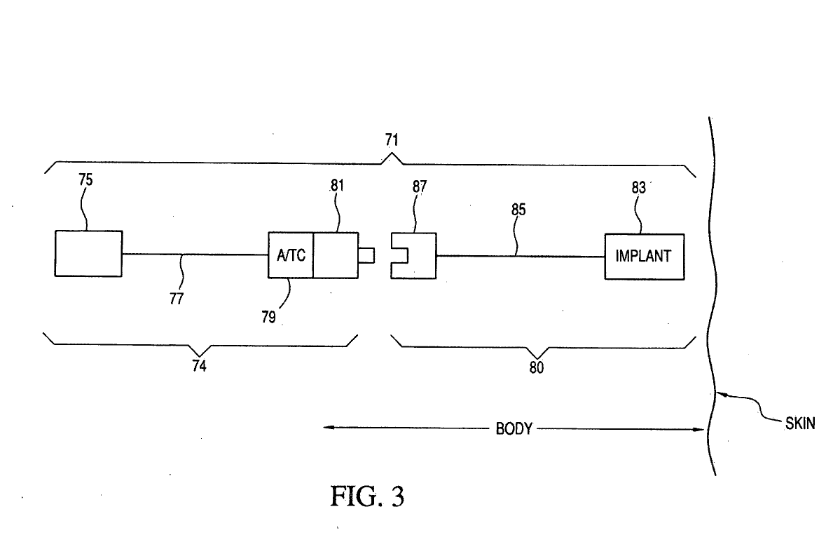

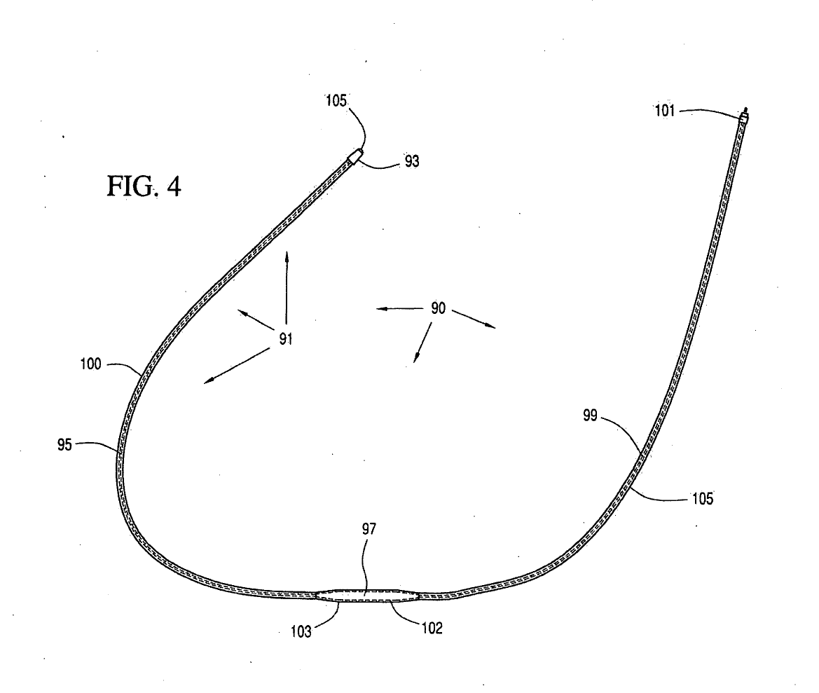

FIG. 3 is a schematic diagram of a preferred embodiment of implantable biotelemetry system 71 of the present invention. The entire system 71 is of a compact size which allows it to be completely implanted within the body 73 of a biological system typically an animal that is under study. The animal can vary from large animals such as cows or a sheep down to dogs and rats. The system typically has a variety of sensors to measure blood pressure, blood flow, and temperature as well as EKG sensors. Standard sensors for flow can be used such as Doppler ultrasound or transit time ultrasound sensors to measure blood flow, etc. Pressure can be measured with piezoresisteive based pressure sensors.

The parts of the implantable biotelemetry system 71 are a sensor assembly 74 and an implant service unit 79. The sensor assembly 74 consists of a sensor 75 which connects by lead 77 to amplifier, temperature compensation and impedance conversion circuitry 79, which in turn connects to connector 81. Sensor 77 circuitry 79 and connector 81 form a unitary liquid impervious sealed unit. The only exposure to the outside environment is senor 75. Implant service unit 79 consists of implant 83 which connects by lead 85 to connector 87. Connectors 81 and 87 are configured to detachably and securely fit together to form a sealed liquid impervious connection.

As noted above implantable biotelemetry system 71 is encased in a liquid impervious sealed package which allows it to be wholly implanted within the body 73 to of a biological subject. As noted above amplifier/temperature compensation circuitry is located at the sensor side within sensor assembly 74 of the implant before connector 81 to thereby condition the signal before it passes through connector 81-87 junction. As noted above this conditions the signal and thus avoids the degradation of the data quality of the signal that a high impedance unconditioned signal could very well experience as it passes through the junction formed by connectors 81-87. This allows for a highly flexible implantable biotelemetry system 71 in which different sensor arrays 74 can be attached or detached to implant service unit 80. Therefore providing the signal generated by sensor 75 with amplification, temperature compensation and impedance conversion before the signal passes through connector 81-87 junction is an important attribute of the present invention. This makes the arrangement of the connector and amplifier/temperature compensation module clearly very crucial in an implantable system.

FIG. 4 is a view of a preferred embodiment of a pressure sensor assembly 90 (unit 74 FIG. 3) of the present invention. It consists of pressure probe 91 with a pressure sensor 93 connected to electrical lead 95. Electrical lead 95 connects to amplifier, temperature compensation and impedance conversion circuitry 97. Electrical lead 99 connects amplifier, temperature compensation and impedance conversion circuitry 97 to connector 101.

Unit 90 is completely sealed with the only exposed part being a surface of sensor 93, in the case of this particular unit, to obtain the appropriate pressure readings. Electrical lead 91 is encased in a liquid impervious highly flexible biocompatible material 100 such as silicon covering. The liquid impervious covering 100 forms a unitary connection to the covering 102 of circuitry 97 a highly flexible liquid impervious biocompatible material such as medical grade silicon. To further protect circuitry 97 it has surrounding the circuitry 97 under the outside silicon layer 102 is shrink wrap tubing 103. Lead 99 similarly has a highly flexible liquid impervious covering 105 that forms a unitary connection with covering 102 of circuitry 97.

The dimensions of pressure sensor unit 90 are as follows: a) sensor 93 is 1.2 mm in diameter and 4 mm in length, b) lead 91 is 20 cm long, c) circuitry unit 97 is 7 mm in diameter and 3 cm long, d) lead 91 is 12.5 cm long and 2.5 mm wide, and e) connector 101 is 3.5 cm long and at its greatest diameter 6 mm in diameter. These sizes have only been provided to give some conception of a possible configuration of unit 90. Those skilled in the art will realize that the dimensions of unit 90 can be made much smaller or larger depending on the application and size of the animal under study. Sensor unit 90 is a highly flexible and pliable unit which allows it to be easily implanted in a subject animal's body without causing discomfort or disruption to the animal.

In the preferred embodiment depicted in FIG. 4 pressure probe 91 is a pressure probe made by Millar Laboratories of Huston Tex. Sensor 93 has a piezoresistive sensor 105. When unit 90 is implanted in a subject animal sensor 93 is inserted in to the specific artery of the subject animal to obtain the desired pressure readings. Amplifier, temperature compensation and impedance converter circuit 97 is standard circuitry well known in the art so a discussion of its specific makeup is not necessary since those skilled in the art would know how to design such circuits. Naturally, the amplifier circuitry would be increasing the strength of the signal and the impedance converter would be decreasing the impedance of the signal. Additionally, those skilled in the art will also know how to miniaturize such circuits on a silicon chip or otherwise so a discussion of how to fabricate them is not necessary for this disclosure.

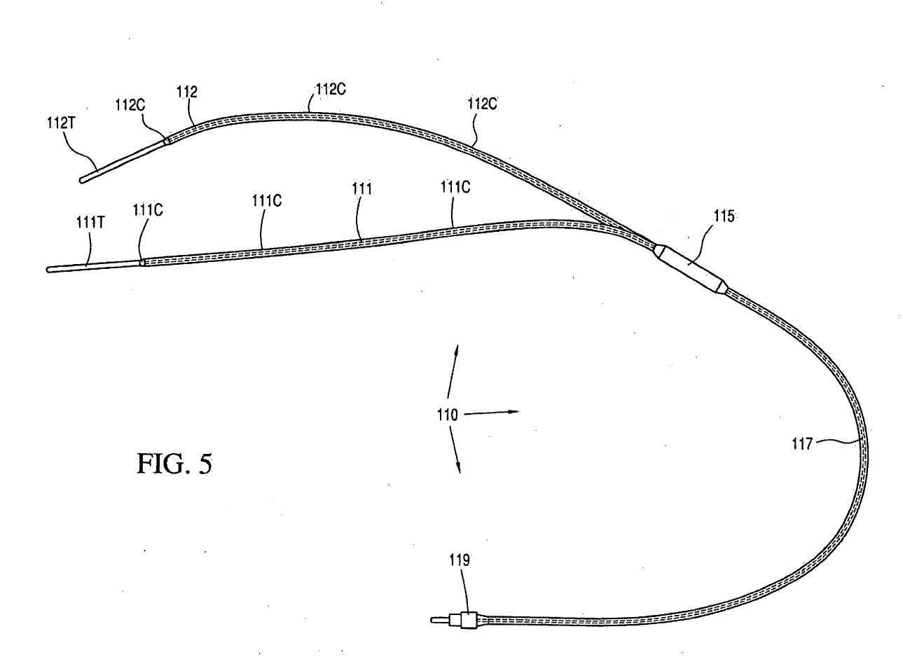

FIG. 5 is an example of an EKG sensor unit 110 made according to the present invention. Unit 110 has similar dimensions to unit 90 of FIG. 4. Electrical leads 111 and 112 connect to amplifier, temperature compensation and impedance conversion circuitry 115 which in turn through lead 117 connects to connector 119. Electrical leads 111 and 112 have tips 111T and 112T with bear wires which can be attached to the appropriate interior portion of the subject animal to obtain signals for monitoring the subject animal with an EKG. Starting at point 111C and 112C and moving towards circuit 115 leads 111 and 112 are covered with a liquid impervious biocompatible covering which is a uniform unbroken covering all the way to connector 119, the same as it is on sensor unit 90. Likewise in all other respects sensor unit 110 is similar to sensor unit 90. Sensor unit 110 is a highly flexible and pliable unit which allows it to be easily implanted in a subject animal's body without causing discomfort or disruption to the animal and its activities.

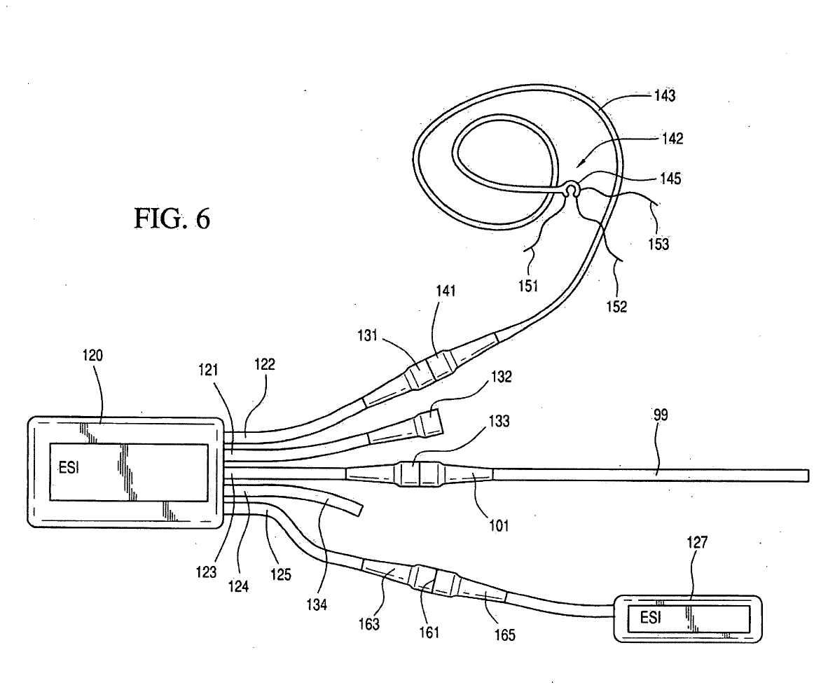

As schematically shown in FIG. 3 sensor unit 71 connects to an implant service unit 80 both of which are wholly implanted in the body 73 of the animal under its skin 72. FIG. 6 is a plan view of a preferred embodiment of a service unit 120 with power pack 127. Service unit 120 contains a microcontroller, wireless transceiver unit, computer memory, other circuitry and software, all of which is not shown to control operation of sensors attached to unit 120. Unit 120 transmits data gathered by the sensors to a separate base station or stations, also not shown. Operation of unit 120 is controlled through the base station. In turn the base station would typically connect to a PC or other computer from which actual control would be maintained. The PC would be running appropriate software to allow the PC user to exert control over unit 120 and its attached sensors, gather the data from the sensors. The software on the computer would also store and analyze the data gathered. The microcontroller, wireless transceiver, memory, other ancillary circuitry and software is not shown or discussed in further detail since all are well known the art and can be implemented in a wide variety of ways.

Unit 120 in addition to lead 125 which connects it to power supply 127 has four other leads 121, 122, 123 and 124 with connectors 131, 132, 133 and 134 respectively. Unit 120 can control and monitor vital functions of the subject animal, not shown, on four different channels with four different sensors. As depicted in FIG. 6 connectors 132 and 134 do not have any sensors units attached to them. Connector 133 is connected to connector 101 of sensor 90. In FIG. 6 only connector 101 and lead 99 of pressure sensor are shown. Service unit 120, electrical lead 125 to power supply 127 and all of the leads 121, 122, 123 and 124 are covered by a liquid impervious layer, such as silicon. Also, connectors 131, 132, 133 and 134 all for a detachable secure liquid impervious connection with the connectors of each of the sensor units that connect to them.

Connected to lead 122 is an ultrasound Doppler sensor 142 for monitoring blood flow. Sensor head 145 is secured around a blood vessel with suture threads 15, 152 and 153. Sensor head 145 connects to connector 141 by electrical lead 143. Connector 141 forms a detachable liquid impervious connection to connector 131 of lead 122. Ultrasound Doppler sensor 142 is covered by an appropriate liquid impervious covering such as silicon.

In one variation of the preferred embodiment service unit 120 is 5 cm long, 2.25 cm wide and 6 mm high. Power unit 127 is 5 cm long and 1.7 cm in diameter, the connectors and all of the connectors and leads to the connectors are 5 cm long. These dimensions can vary depending on the application. In the preferred embodiment lead 125 has a connector junction 161 formed by connectors 163 and 165 which form a detachable but secure and liquid impervious connection.

Thus, as described the present invention provides a modular compact and fully implantable biotelemetric monitoring system.

While the invention has been particularly shown and described with reference to a preferred embodiment thereof, it will be understood by those skilled in the art that various changes in form and detail may be made to it without departing from the spirit and scope of the invention.

I claim:

1. An implantable telemetric sensor system comprising:
a. a sensor assembly including a sensor, signal conditioning circuitry and a connector all in a sealed liquid impervious package;
b. an implant service package with a power supply to power the implant service unit, a microcontroller to control operation of the implant service unit, memory for storing software to run the implant service unit with the microcontroller, a transceiver for communicating with a base unit and a connector capable of forming a liquid impervious detachable electrical connection with said sensor connector of said sensor assembly; and
c. wherein when said sensor assembly is implanted inside a living biological system and said sensor is connected to monitor a function of the biological system and said connector is attached to a connector of said implant service package.

2. The implant of claim 1 wherein said conditioning circuitry includes circuitry for amplifying signals generated by said sensor.

3. The implant of claim 1 wherein said conditioning circuitry includes temperature compensating circuitry

4. The implant of claim 1 wherein said conditioning circuitry includes circuitry for changing the impedance of signals generated by said sensor.

5. The implant of claim 1 wherein said sensor assembly is pressure sensor that uses a piezoresistive surface for sensing pressure.

6. An implantable sensor assembly comprising:
a. a sensor in electronic communication with conditioning circuitry and said conditioning circuitry is electrical communication with a connector, and wherein said sensor, said conditioning circuitry and said connector are joint as one unit in a liquid impervious sealed package; and
b. said connector of said sensor assembly is configured to form a detachable liquid impervious detachable connection with a connector of an implant service unit.

7. The implant of claim 6 wherein said conditioning circuitry includes circuitry for amplifying signals generated by said sensor.

8. The implant of claim 6 wherein said conditioning circuitry includes temperature compensating circuitry

9. The implant of claim 6 wherein said conditioning circuitry includes circuitry for changing the impedance of signals generated by said sensor.

10. The implant of claim 6 wherein said sensor assembly is pressure sensor that uses a piezoresistive surface for sensing pressure.
